(19) United States
(12) Patent Application Publication
(10) Pub. No.: US 2026/0198824 A1
(43) Pub. Date: Jul. 16, 2026

(54) SYSTEM AND METHOD FOR MENTAL HEALTH MONITORING

(71) Applicant: Innsightful, Inc., Ann Arbor, MI (US)

(72) Inventors: Farzad Ehsani, Sunnyvale, CA (US); Demitrios L. Master, Cupertino, CA (US)

(21) Appl. No.: 19/449,173

(22) Filed: Jan. 14, 2026

Related U.S. Application Data
(60) Provisional application No. 63/745,174, filed on Jan. 14, 2025.

(51) Int. Cl. A61B 5/16; A61B 5/00; A61B 5/0205; A61B 5/021; A61B 5/024; A61B 5/0531; A61B 5/08; G16H 40/67

(52) U.S. Cl. CPC A61B 5/165; A61B 5/02055; A61B 5/7264; A61B 5/7278; G16H 40/67; A61B 5/021; A61B 5/02405; A61B 5/02416; A61B 5/0531; A61B 5/0816; A61B 5/681

(57) ABSTRACT

A system and method for mental health monitoring and intervention. The method includes generating one or more biometric data sets from measuring and/or estimating one or more physiological markers via one or more biosensors and extracting features from the one or more biometric data sets. The method includes executing parallel multi-temporal machine learning modeling using the extracted features as inputs, whereby the multi-temporal modelling includes: a pipeline for one or more short-term models adapted for detecting acute stress responses; and a pipeline for one or more long-term models for detecting chronic mental health conditions.

### CROSS-REFERENCE TO RELATED APPLICATIONS

The present application claims priority to U.S. Provisional Application No. 63/745,174 filed Jan. 14, 2025, and incorporated herein by reference in its entirety.

### FIELD

The present teachings relate to systems and methods for mental health monitoring, using biometric sensing and machine learning techniques.

### BACKGROUND

Chronic anxiety, stress, and depression represent a rapidly growing healthcare challenge in the United States. In 2022 alone, 49.6 million U.S. adults and 3.7 million U.S. youth experienced mental illness, yet less than half received treatment. Lifetime prevalence of anxiety disorders in the U.S. is approximately 28.8%. On average, 1 out of every 4 adults have experienced at least one anxiety disorder.

Anxiety disorders not only affect patient health, but also have a negative economic impact on the healthcare system, with a total annual cost estimated at almost $47 billion. More than 75% of the cost can be attributed to morbidity, mortality, lost productivity, and other indirect costs.

High cost, a lack of providers, transportation issues, and stigma are the primary reasons people are forced to suffer with mental illness. Left untreated, mental illness often results in lower education levels, lower income, increased unemployment, somatic disorders, loss of executive function, and even suicide.

College students are increasingly impacted by anxiety, with over 50% suffering from anxiety and psychological distress. Key drivers include academic performance, pressure to succeed, and post-graduation uncertainty. Depression and anxiety are the top two mental health concerns for college students, with current depression and anxiety rates at 44.2% and 36.6%, respectively. Between 25% and 33% of students meet the criteria for anxiety or depressive illness during their college careers. Additionally, student use of college counseling centers increased by 30 to 40% from 2009 to 2015. These numbers are expected to increase; 9.4% of children (3 to 17 years in age) have been diagnosed with anxiety, and 4.4% have been diagnosed with depression. Yet, with these increases, only 15% of students with self-identified mental health challenges engaged in college-offered counseling in 2022-2023. Forty-two percent seek support from friends, and over 30% have no support mechanism. Studies have found that 64% of students quit their studies because of mental health issues. Almost half of those students did not use mental health services.

The most common treatments for anxiety and stress today are drugs and psychotherapy, which are often used effectively in combination. A well-known effective type of therapy is cognitive behavioral therapy (CBT), which specifically trains patients to recognize anxiety triggers and change their response patterns to reduce overall anxiety—but it is typically expensive. It also requires a commitment to the process. Pharmaceutical interventions for anxiety often include antidepressants, such as serotonin reuptake inhibitors, or a mixed-mechanism antidepressant. Anxiolytics have also been used to treat anxiety disorders, but they are no longer considered a first option due to significant side effects. Studies have shown that those with anxiety disorders are more prone to drug addiction and abuse, including opioid addiction. Therefore, it would be desirable to minimize dependence on pharmaceutical interventions.

Paths to access these treatments are under strain. Despite the roughly 20% annual job growth rate for mental health counseling, there remains limitations in the healthcare system. In 2018, one-third of all college counseling centers had a waiting list of up to 35 weeks. Kaiser Family Foundation data indicates more than 500 additional psychiatrists are needed to eliminate the current shortfall nationwide. In 2021, 69% of university Counseling and Psychological Services (CAPS) centers reported difficulty recruiting for an open position. Of those, larger schools struggled most —90% of schools of 30,000 to 35,000 students reported difficulty recruiting.

In the College Pulse Survey 2021, 25% of college students said their college should prioritize funding a 24-hour emergency mental health hotline if additional funds are available, and 20% said new or expanded tele-counseling services are needed. One recent survey of postsecondary educators found that nearly 80% believe emotional well-being is a “very” or “extremely” important factor in student success. Yet, young people are also often reluctant to seek treatment due to social and self-stigmatizing attitudes towards mental health interventions. Most students worry about negative labeling by peers, and discriminatory reactions from peers. They also worry about compromised confidentiality. The privacy of online care delivery can reduce or eliminate these concerns.

Recently, the availability of remote interventions, for example Internet- and mobile-based interventions, has increased, including therapy via telemedicine and stand-alone digital health apps. Online delivery of mental health care is an easily scalable and non-resource-intensive solution to address student mental health challenges. Online delivery of care lessens the stigma of receiving care, while making it more accessible at a relatively low cost. A recent study published in the Journal of the American Medical Association found that two-thirds of adults preferred to have at least some video visits; 25% of young adults said they prefer telehealth to in-person visits. An asynchronous care delivery format also aligns with students' busy lifestyles. Using digital tools to successfully deliver personalized, evidenced-based therapy while keeping clinicians in the loop can have a significant positive impact on student mental health on college campuses.

Early intervention is critical to positive change. Current literature suggests that preventing anxiety from becoming chronic, irreversible, or preventing it from developing into a comorbidity with depression is crucial. Studies show real-time detection of anxiety can prevent negative episodes at work, school, and home. It can also lessen the severity of anxiety, as well as depression, while improving symptoms.

Self-report measures are one diagnostic tool to identify the presence and severity of various symptoms, and have been clinically validated to correlate with various anxiety disorders. These self-report measures are in the form of questionnaires that produce a score on a scale (e.g., normal, moderate, or severe). Exemplary self-report measures include Perceived Stress Scale (PSS-10), Generalized Anxiety Disorder (GAD-7), Patient Health Questionnaire (PHQ-9), Brief Resilience Scale (BRS-6), Posttraumatic Stress Disorder Checklist (PCL-5), and Somatic Symptom Scale (SSS-8). These measures are not conventionally employed alone for mental health assessment but are typically one facet of a clinical approach to assessment, such as providing an initial baseline and to compare to that baseline over time.

Current efforts at using biometric sensing for anxiety disorder assessment report weak correlations to self-report measures. See, for example, Velmovitsky et al., Can heart rate variability data from the Apple Watch electrocardiogram quantify stress?, Front. Public Health 11:1178491, Jul. 5, 2023. Even if strong correlations between biometric data and just one self-report measure were to be established, what is needed is a robust system and method that establishes strong correlations with most, if not all self-report measures, in order to accurately identify a multitude of symptoms or disorders related to anxiety.

Challenges in obtaining these strong correlations relate to a multitude of factors having an effect on biometric signals including physiological factors (e.g., age, weight, gender, and pre-existing conditions), behavioral factors (e.g., activity levels, sleep habits, occupation, and recreational habits), and even psychological factors (e.g., existing psychological disorders).

Machine learning relating to biometric sensing has been investigated with mixed results and as-of-yet, weak statistical power due to small data sets. Vos et al., Generalizable machine learning for stress monitoring from wearable devices: A systematic literature review, Int. J. Med. Inform. 173(2023) 105026. A number of studies in this review relied upon data from medical-grade devices, which impose limitations on use compared to widely-available consumer devices with on-board biometric sensing. Another limiting factor noted in the review is the need for data labelling to characterize stress or no-stress, or scoring on a range, for data collected data to be useful in supervised machine learning.

In general, existing data analysis methods in this field are insufficient to reliably detect anxiety disorders or symptoms of anxiety disorders based on biometric sensing, particularly biometric sensing using commercial products such as mobile phones, smart wearables, or the like. This insufficiency further limits reliable real-time or near real-time data analysis that would lend to early interventions of acute stress responses.

It would be desirable to provide a system and method that monitors acute stress responses and chronic mental health conditions, where the acute stress responses are monitored in real-time or near real-time and chronic mental health conditions are detected in a short timeline (e.g., in a matter of 1 to 12 weeks or more preferably 2 to 4 weeks) to provide for early interventions.

It would be desirable to provide a system and method that utilizes biometric sensing available on common consumer devices such as mobile phones and smart wearables.

It would be desirable to provide a system and method that analyzes data via machine learning techniques, and without the need for data labelling, or at least with a reduced need for data labelling relative to conventional techniques.

It would be desirable to provide a system and method that produces strong correlations between biometric data and clinical measures, such as self-report measures.

### SUMMARY

The present teachings describe a method for mental health treatment, which may address at least some of the needs identified above. The method may comprise generating one or more biometric data sets from measuring and/or estimating one or more physiological markers via one or more biosensors. The method may comprise extracting features from the one or more biometric data sets. The method may comprise executing parallel multi-temporal machine learning modeling using the extracted features as inputs, whereby the multi-temporal modelling includes: a pipeline for one or more short-term models adapted for detecting acute stress responses; a pipeline for hourly models adapted for detecting pain, stress, exhaustion, and mood fluctuations; a pipeline for daily models monitoring sleep quality, gastrointestinal issues, and overall mood; and a pipeline for long-term models assessing chronic mental health conditions.

The one or more physiological markers may include heart rate, heart rate variability, electrodermal activity, respiratory activity, body temperature, body movement, blood oxygen, blood pressure, or any combination thereof. Each of the one or more biometric data sets may correspond with one of the one or more physiological markers.

The one or more physiological markers may include at least heart rate variability.

The feature extraction for the heart rate may include statistical features including mean heart rate and standard deviation of heart rate.

The feature extraction for the heart rate variability may include statistical features, time-domain features, frequency-domain features, and nonlinear features. The statistical features may include mean heart rate variability and standard deviation of heart rate variability. The time-domain features may include standard deviation of NN intervals and root mean square of successive differences. The frequency-domain features may include normalized low-frequency, normalized high-frequency, and a ratio of normalized low-frequency to normalized high-frequency.

The nonlinear features may be extracted by entropy analysis, nonlinear dynamic analysis, Poincaré plot analysis, second order data differential plotting, and chaotic modeling.

The feature extraction for the electrodermal activity may account for both tonic and phasic components. The features may include skin conductance level, skin conductance response, and a correlation coefficient between skin conductance level and time.

The features for the respiratory activity may include breath rate and respiratory sinus arrhythmia.

The features for the body temperature may include mean temperature, standard deviation of temperature, and differential temperature over a time period.

The features for the blood pressure may include a mean of blood pressure, a standard deviation of blood pressure, and differential blood pressure measured on a periodic basis.

The method may further comprise, before the feature extraction, performing data parameterization of at least the heart rate variability including applying a moving average technique.

The long-term models may include Random Forests, Gradient Boosting Machines, Support Vector Machines, and Multi-layer Perceptrons.

The short-term models may include gradient boosting machines and support vector machines. The short-term models may employ ensemble learning techniques.

The method may further comprise administering one or more self-report measures.

The method may further comprise, before the feature extraction, data conditioning of the one or more physiological markers. The data conditioning may include: implementing piecewise linear, spline, and regression-based interpolation for missing values; performing adaptive filtering for noise reduction specific to each type of the one or more physiological markers; and normalizing sampling frequencies accounting for different sampling frequencies of the one or more biosensors.

The method may further comprise generating interventional recommendations based on the detected acute stress responses and/or the chronic mental health conditions.

The present teachings describe a system, which may address at least some of the needs identified above. The system may comprise a sensor network comprising one or more biosensors. The system may comprise a user device comprising one or more processors and one or more non-transitory memory storage mediums storing computer-readable instructions that, when executed, detect acute stress responses and/or chronic mental health conditions. The system may comprise a remote processing platform comprising one or more processors and one or more non-transitory memory storage mediums storing computer-readable instructions that, when executed, detect acute stress responses and/or detect chronic mental health conditions.

The one or more biosensors may include at least a photoplethysmography sensor.

The one or more biosensors may include one or more photoplethysmography (PPG) sensors, temperature sensors (e.g., thermocouples, resistance temperature detectors (e.g., negative temperature coefficient sensors), thermistors, or semiconductor-based integrated circuits), electrodermal activity (EDA) sensors, pulse oximeters, accelerometers, gyroscopes, or any combination thereof.

The sensor network may be on-board a smart wearable device.

### BRIEF DESCRIPTIONS OF THE DRAWINGS

FIG. 1 is a block diagram illustrating an exemplary system according to the present teachings.

FIG. 2 is a table showing exemplary physiological marker features according to the present teachings.

FIG. 3 is a flowchart depicting an exemplary method according to the present teachings.

FIG. 4 is a flowchart depicting an exemplary method according to the present teachings.

### DESCRIPTION

### Introduction

The present application sets forth an improved system and method for mental health monitoring. In one aspect, real-time (in a time-frame of about 1 second to about 60 seconds) or near real-time (in a time-frame of about 1 minute to about 10 minutes) mental health monitoring may be achieved to detect acute stress responses. In another aspect, chronic mental health conditions may be detected over an appropriate time period, as discussed herein. The detection of both acute stress responses and chronic mental health conditions may be referred to as multi-temporal mental health monitoring. That is, monitoring both on a short time scale (a few hours or even a few days for acute stress response) and a long time scale (2 or more weeks for chronic mental health conditions).

The mental health monitoring system and method described herein provides solutions for evidence-based mental health assessment and continuous biometric-driven symptom detection. The system and method incorporate unique multi-modal machine learning architectures, real-time physiological signal processing, and sophisticated clinical assessment integration mechanisms, enabling comprehensive mental health monitoring while maintaining clinical validity and reducing assessment delays.

The system and method address the challenges of balancing automated symptom detection with evidence-based clinical assessment while ensuring timely intervention. It offers a versatile solution that combines advanced biometric processing with validated clinical measures (self-report measures), potentially improving mental health outcomes across different temporal scales and symptom types. The invention's ability to provide continuous monitoring capabilities while maintaining clinical assessment accuracy represents a significant advancement in mental health technology.

Acute stress response may be related to acute symptoms such as pain, gastrointestinal issues (e.g., stomachache, constipation, or the like), exhaustion, cravings, focus, mood, acute stress, or any combination thereof. Acute stress responses may be detected in the short-term and may be detected via short-term models described herein.

Chronic mental health conditions, as described herein, may refer to anxiety, depression, chronic stress, burnout, post-traumatic stress disorder (PTSD), or any combination thereof. Chronic mental health conditions may be detected on a long-term basis (e.g., 2 or more weeks), which may be aligned with the self-report measure associated with each chronic mental health condition. Anxiety may be measured by GAD-7, which accounts for a 2-week period. Depression may be measured by PHQ-9, which accounts for a 2-week period. Chronic stress may be measured by PSS-10, which accounts for a 2-week period. PTSD may be measured by PCL-5, which accounts for a 4-week period. Somatic symptoms may be measured by SSS-8, which accounts for a 1-week period. Resilience may be measured by BRS-6, which does not have a specific associated period.

The method of the present teachings may also account for hourly symptoms and daily symptoms in the detection of chronic mental health conditions. Acute symptoms may include stress, pain, exhaustion, and mood fluctuations. Acute symptoms may also include gastrointestinal issues. Acute symptoms can be detected in as little as 5-minute intervals, although many symptoms fluctuate only over longer time periods. Daily symptoms, such as sleep quality or overall mood, may be detected over a period of 12 hours or more.

As will be discussed in more detail interventions may be provided to users, in response to indications from the method described herein, to manage stress, for example, when acute stress responses are detected. So, while detection and continued monitoring of chronic mental health conditions is underway, users can be provided with tools for managing stress. For example, when preparing for a task that requires significant physical and cognitive resources.

By measuring existing stress, pain, and fatigue (for physical wellbeing) and resilience and mood (for cognitive wellbeing), the system and method of the present teachings can clearly show individuals when their physical or cognitive “battery” will run out, and when they need to recharge.

The system and method can be deployed in clinical settings for patient monitoring, educational institutions for student mental health support, and corporate environments for employee wellness programs. This technology is particularly valuable for remote patient monitoring, enabling healthcare providers to track mental health symptoms continuously without requiring frequent in-person visits. The system's ability to detect both acute stress and chronic mental health conditions makes it suitable for preventive healthcare, psychiatric practices, and occupational health services. The scalable architecture allows for widescale deployment across various healthcare delivery platforms.

### System

The present teachings describe a data analysis method unique to the field of mental health monitoring using biometric sensing, particularly biometric sensing via sensors on-board common commercial computing devices such as mobile devices (e.g., mobile phones, tablets, and smart wearables), laptop computers, or desktop computers. Said data analysis method has been found to provide both acute stress response detection and chronic mental health condition detection that correlates strongly to actual symptoms and self-report measures.

The biometric sensors of the present teachings may include sensors adapted for measuring and/or estimating heart rate, heart rate variability, respiratory rate, temperature, body movement, blood oxygen, blood pressure, electrodermal activity, or any combination thereof. The present teachings contemplate that one or more of these physiological markers may be measured and/or estimated from one or a combination of biometric sensors. The biometric sensors may include one or more photoplethysmography (PPG) sensors, temperature sensors (e.g., thermocouples, resistance temperature detectors (e.g., negative temperature coefficient sensors), thermistors, or semiconductor-based integrated circuits), electrodermal activity (EDA) sensors, pulse oximeters, accelerometers, gyroscopes, or any combination thereof. In some exemplary aspects of the present teachings, a plurality of biometric sensors may be used, which may be referred to as a sensor network.

Smart wearables, as discussed herein may be any wearable device with an on-board biometric sensor. Smart wearables may include, but are not limited to, smart watches, smart rings, heart rate monitors, or the like.

The computing devices described herein may include one or more network modules to wirelessly communicate with one or more other computing devices over a network. The network module may operate according to IEEE 802.15.1 (commonly referred to as Bluetooth). The network module may operate according to IEEE 802.11 (commonly referred to as WiFi). The present teachings also contemplate a wired connection providing for communication.

The system of the present teachings may comprise one or more user devices. The user device may include one or more processors, one or more memory storage mediums (e.g., non-transitory memory storage mediums), one or more network modules, or any combination thereof. Computer-readable instructions may be stored in the memory storage medium. The processor may execute the computer-readable instructions to receive and process the physiological marker data from the sensor network, manage local data storage, handle communications with a remote platform, control I/O devices, or any combination thereof. The user device may be a mobile phone, a tablet, a laptop computer, or a desktop computer.

The system of the present teachings may comprise one or more input/output (I/O) devices. The I/O device may be coupled to the user device. The I/O device may facilitate user interaction with the system. The I/O device may include a display screen for presenting information, queries, and results to the user. The I/O device may include one or more input mechanisms (e.g., touchscreen, keyboard, microphone) for receiving user responses to queries and other inputs. The I/O device may enable delivery of self-report measures and collection of user feedback regarding their mental state.

The system of the present teachings may comprise one or more remote processing platforms. The remote processing platform may include one or more processors, one or more memory storage mediums (e.g., non-transitory memory storage mediums), one or more network modules, or any combination thereof. The remote processing platform may communicate with the user device over a network connection. The processor may execute computer-readable instructions to receive biometric sensor data and/or user inputs, process this information using trained machine learning models, and return mental health assessments and interventional recommendations. The remote processing platform provides sophisticated data analysis while reducing computational burden on the user device.

### Method

The method may comprise one or more of the following steps. Some of the steps may be duplicated, removed or eliminated, rearranged relative to other steps, combined into one or more steps, separated into two or more steps, or a combination thereof.

Note that any of the method steps described herein may be performed by hardware, software, or any combination of these approaches. For example, a non-transitory computer-readable storage medium may store thereon instructions that when executed by a computing device result in performance according to any of the embodiments described herein.

The method may comprise data conditioning. The data conditioning may include missing value imputation, outlier removal, signal resampling, or any combination thereof.

Missing value imputation may include interpolation. The interpolation may prioritize temporal proximity, utilizing nearest available segments on the timeline while minimizing reliance on future data points. The interpolation may include piecewise linear, spline, or regression-based approaches, or any combination thereof, with least-square or other types of optimization.

The method may comprise data parameterization. The data parameterization may include specific sampling frequency normalization. Moving average techniques may be employed.

The method may comprise feature extraction. The feature extraction may be tailored to each of the physiological markers, to select features that lend to robust and accurate acute stress response detection and chronic mental health condition detection.

Methods for heart rate may include linear feature extraction methods. The features may include mean heart rate (mHR), standard deviation of heart rate (SDHR), or both.

Methods for heart rate variability (HRV) may include linear and/or nonlinear feature extraction methods. Preferably at least nonlinear feature extraction methods. Linear feature extraction methods may include: statistical features (e.g., mean, median, standard deviation, or any combination thereof), time-domain features (e.g., standard deviation of NN intervals (SDNN), root mean square of successive differences (RMSSD), or both), frequency-domain features (e.g., normalized low-frequency (Norm-LF), normalized high-frequency (Norm-HF) components, a ratio of Norm-LF to Norm-HF, or any combination thereof). Nonlinear feature extraction methods may include: entropy analysis, nonlinear dynamic analysis, Poincaré plot analysis, second order data differential plotting; chaotic modeling, or any combination thereof.

Features for electrodermal activity (EDA) include skin conductance level (SCL), skin conductance response (SCR), a correlation coefficient (Cor-SCL) between skin conductance level and time, or any combination thereof.

Features for respiratory data may include breath rate, respiratory sinus arrhythmia, or both.

Features for body temperature may include mean temperature (m-Temp), standard deviation of temperature (SD-Temp), differential temperature measurements over a time period (e.g., every 5 minutes), or any combination thereof.

Features for body movement include X, Y, and Z axis values. These values may be used to define: the features of activity state classifications (e.g., rest, walking, running), motion patterns, motion above defined thresholds, or any combination thereof.

The blood oxygen features may include continuous blood oxygen saturation measurements, averaged on a periodic basis (e.g., every minute).

The blood pressure features may include mean blood pressure (M-BP), standard deviation of blood pressure (SD-BP) measured on a periodic basis (e.g., hourly), differential blood pressure measured on a periodic basis (e.g., taken every 30 minutes), or any combination thereof.

The method may comprise data modelling. The data modeling may employ ensemble classifiers for acute stress detection. The method may employ hierarchical models for assessing chronic mental health conditions.

The model architectures may include Random Forest, Gradient Boosting Machines, Support Vector Machines, Multi-layer Perceptrons, and neural nets (MPC, Convolutional, RNN, Transformers, temporal neural network) or any combination thereof.

The method may comprise model tuning. The method may comprise feedback and optimization loops.

The method may comprise administering one or more self-report measures. The self-report measures may include Perceived Stress Scale (PSS-10), Generalized Anxiety Disorder (GAD-7), Patient Health Questionnaire (PHQ-9), Brief Resilience Scale (BRS-6), Posttraumatic Stress Disorder Checklist (PCL-5), Somatic Symptom Scale (SSS-8), or any combination thereof.

### EXAMPLES

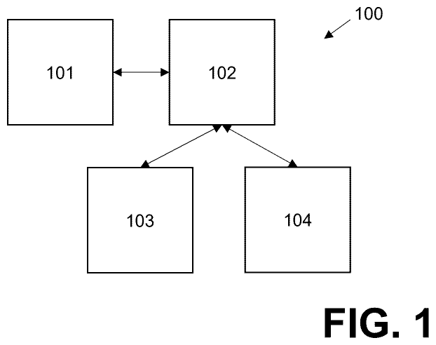

Referring to FIG. 1, an exemplary system 100 according to one aspect of the self-report present teachings includes a sensor network 101, a user device 102, a remote processing platform 103, and input/output (I/O) devices 104. The system 100 provides continuous monitoring and assessment of mental health conditions through integration of physiological marker measurements and/or estimations and user inputs.

The sensor network 101 comprises a biometric sensor or more preferably a plurality of biometric sensors, which are configured to continuously collect physiological marker data from a user. The biometric sensors may be integrated into a wearable or other computing device described herein.

The sensor network 101 can include a photoplethysmography (PPG) sensor for measuring heart rate and blood flow patterns, an electrodermal activity (EDA) sensor for detecting changes in skin conductance associated with, e.g., emotional and stress responses, a pulse oximeter (SpO2) sensor for monitoring blood oxygen saturation, a temperature sensor for tracking body temperature, and an accelerometer for detecting body movement.

The user device 102 includes one or more processors, one or more memory storage mediums, and computer-readable instructions stored in the one or more memory storage mediums. The user device 102 may be a mobile phone, a tablet, a laptop computer, or a desktop computer. The processor executes the computer-readable instructions to receive and process the physiological marker data obtained from the sensor network 101, manage local data storage, handle communications with the remote platform 103, and communicate with and control the I/O devices 104.

The I/O devices 104 are coupled to the user device 102 and facilitate user interaction with the system 100. The I/O devices 104 devices include a display screen for presenting information, queries, and results to the user, and input mechanisms (e.g., touchscreen, keyboard, microphone) for receiving user responses to queries and other inputs. The I/O devices 104 enable delivery of self-report measures and collection of user feedback regarding their mental state.

The remote processing platform 103 includes one or more processors, one or more memory storage mediums, and computer-readable instructions implementing machine learning models and other analytics capabilities. The remote processing platform 103 communicates with the user device 102 over a network connection, which may be wired or wireless, to receive physiological marker data and user inputs, process this information using trained machine learning models, and return mental health assessments and interventional recommendations. The remote processing platform 103 enables sophisticated data analysis while reducing computational burden on the user device 102.

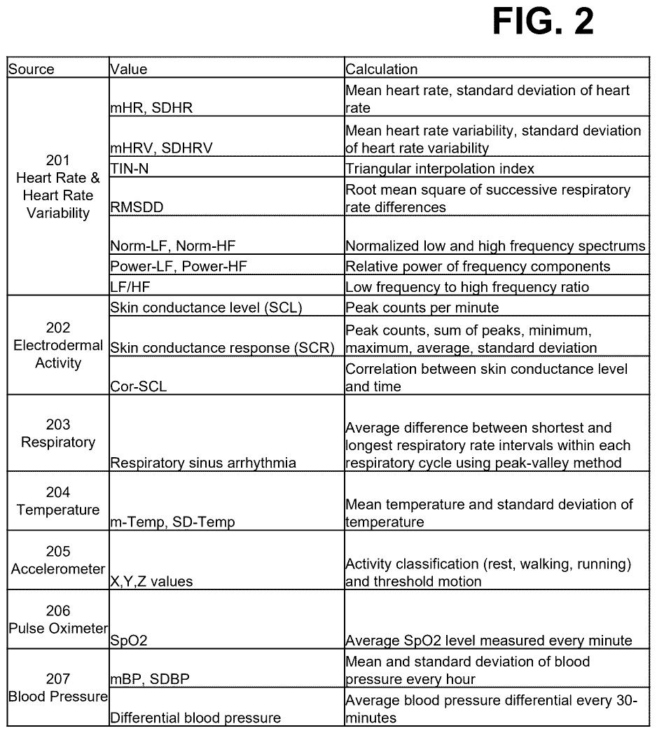

Referring to FIG. 2, the exemplary physiological marker data inputs collected by the system 100 comprise a plurality of different types of physiological marker measurements and/or estimations that are used for the mental health assessment described herein. Diverse data streams provide for comprehensive monitoring of the user's physiological state and its correlation with acute stress responses and chronic mental health conditions across different temporal scales.

It is understood by the present teachings that calculations may be performed on raw sensor data to obtain a desired value that is meaningful for analysis, which may also be referred to herein as feature extraction. These values have been found to contribute to the robust mental health assessment described herein. That is, they have been found to reveal useful information about physiological responses relevant to the stress and mental health assessments of the present teachings, particularly with the machine learning pipelines described herein.

The heart rate and heart rate variability data 201 encompasses multiple derived metrics that provide insights into autonomic nervous system function. Linear feature extraction methods are implemented on heart rate, where features include mean heart rate (mHR) and standard deviation of heart rate (SDHR).

Linear and nonlinear feature extraction methods are implemented on heart rate variability (HRV). Linear feature extraction methods include: statistical features (e.g., mean, median, and standard deviation), time-domain features (e.g., standard deviation of NN intervals (SDNN) and root mean square of successive differences (RMSSD)), and frequency-domain features (e.g., normalized low-frequency (Norm-LF) and normalized high-frequency (Norm-HF) components, and a ratio of Norm-LF to Norm-HF; which serves as an indicator of sympathetic-parasympathetic balance). These measurements are particularly valuable for assessing stress responses and anxiety levels. Nonlinear feature extraction methods include: entropy analysis incorporating sample entropy and approximate entropy for complexity assessment; nonlinear dynamic analysis utilizing Lyapunov exponents and correlation dimension; Poincaré plot analysis examining SD1 (the standard deviation of points perpendicular to the line of identity x=y), SD2 (the standard deviation of points along the line of identity x=y), and their ratio; second order data differential plotting; and chaotic modeling analyzing correlation entropy and fractal dimension.

For the electrodermal activity data 202, the feature extraction process accounts for both tonic and phasic components and analyzes both amplitude and temporal characteristics of skin conductance responses. The features include skin conductance level (SCL), skin conductance response (SCR), and a correlation coefficient (Cor-SCL) between skin conductance level and time. The system 100 tracks peak counts per minute, sum of peaks, and statistical measures including minimum, maximum, average, and standard deviation of skin conductance. The correlation coefficient (Cor-SCL) between skin conductance level and time is calculated to identify trending patterns. These measurements can be particularly relevant for detecting emotional arousal and stress responses. These features are computed within defined temporal windows, creating feature vectors that capture both immediate physiological responses and longer-term patterns.

The respiratory data 203 features which can be derived directly from a respiration signal or from beat to beat intervals-can include breathing rate, calculated as the number of respiratory cycles per minute, and respiratory sinus arrhythmia (RSA), often quantified using a peak-valley method as the average difference between the shortest and longest R-R intervals (or max-min heart rate) within each respiratory cycle. These measures can help characterize breathing and autonomic patterns that are often associated with stress and anxiety. The body temperature data 204 features include mean temperature (m-Temp), standard deviation of temperature (SD-Temp), and differential temperature measurements over a time period (e.g., every 5 minutes). The differential temperature measurements provide insights into physiological stress responses and circadian rhythm patterns that may correlate with mental health states.

The body movement (accelerometer) data 205 X, Y, and Z axis values are used to define the features of activity state classifications (e.g., rest, walking, running), motion patterns, and motion above defined thresholds. This body movement data helps contextualize other physiological measurements and identify behavioral patterns relevant to mental health assessment.

The blood oxygen (pulse oximeter) data 206 features include continuous blood oxygen saturation measurements, averaged on a periodic basis (e.g., every minute). This data stream helps identify potential sleep-related issues and physiological stress responses that may impact mental health.

The blood pressure data 207 features include mean blood pressure (M-BP), standard deviation of blood pressure (SD-BP) measured on a periodic basis (e.g., hourly), and differential blood pressure measured on a periodic basis (e.g., taken every 30 minutes). These measurements provide additional context for cardiovascular responses to stress and anxiety.

Each data stream undergoes specific preprocessing steps to ensure data quality and temporal alignment. The system 100 applies appropriate filtering techniques to remove artifacts and normalize sampling rates across different physiological marker measurements. This preprocessed data serves as input to both the real-time acute stress response models and the long-term mental health condition algorithms, enabling comprehensive monitoring across multiple temporal scales while maintaining clinical relevance.

The integration of these diverse physiological marker measurements enables the system 100 to capture both acute stress responses and long-term patterns indicative of various chronic mental health conditions, providing a robust foundation for evidence-based mental health monitoring and intervention.

This distributed architecture allows the system 100 to combine real-time monitoring through local processing with more complex analytics performed remotely, enabling comprehensive mental health assessment across multiple temporal scales.

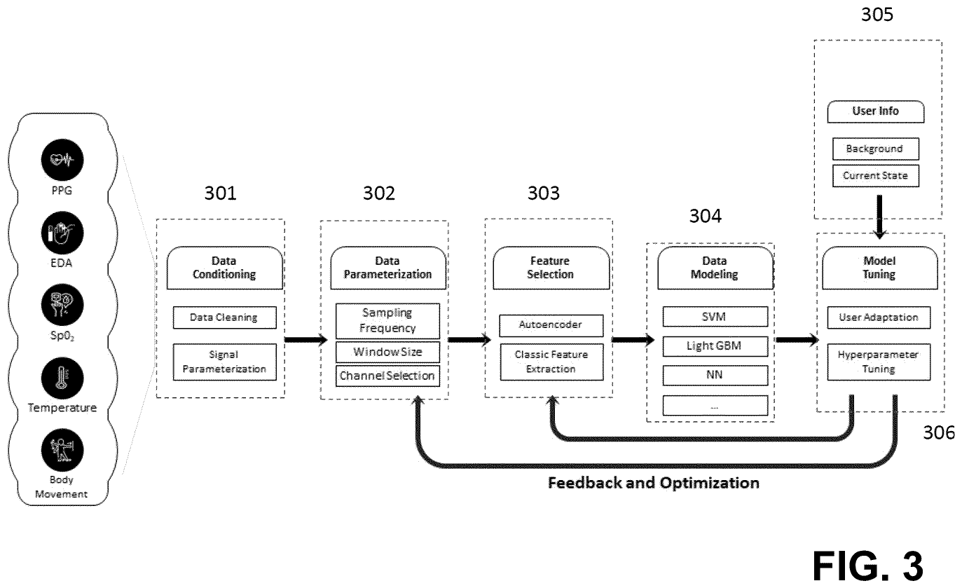

Referring to FIG. 3, the system 100 implements a sophisticated data processing and machine learning pipeline for transforming raw physiological marker measurements into clinically meaningful mental health assessments. The pipeline comprises several key stages: data conditioning 301, data parameterization 302, feature selection 303, data modeling 304, and model tuning 305, with feedback and optimization loops 306 interconnecting these stages.

The data conditioning stage 301 implements preprocessing steps for ensuring data quality and reliability. This stage addresses three challenges in bio signal processing: missing value imputation, outlier removal, and signal resampling.

For missing values, the system 100 employs an advanced interpolation strategy that prioritizes temporal proximity, utilizing nearest available segments on the timeline while minimizing reliance on future data points. The interpolation methodology incorporates piecewise linear, spline, and regression-based approaches with least-square optimization.

In cases where data gaps exceed defined thresholds, the system 100 implements a sophisticated fallback mechanism using default values combined with missing data indicators. This enables downstream components to adapt their processing accordingly. For such scenarios, the system 100 activates secondary pull-back models specifically designed to operate with reduced input feature sets, maintaining operational capability even when certain biometric data is unavailable.

The data parameterization stage 302 transforms the conditioned signals into structured representations suitable for machine learning analysis. This stage implements specific sampling frequency normalization, particularly advantageous for signals like electrodermal activity (EDA) collected at varying frequencies (1-8 Hz) and non-uniform R-R intervals. The system 100 applies moving average techniques with configurable window sizes (e.g., 20 seconds for EDA) to extract both tonic and phasic components of physiological markers.

The feature extraction stage 303 employs multiple approaches for extracting meaningful features from the parameterized data, as described above with regard to FIG. 2.

The feature extraction process is optimized for different temporal windows corresponding to the intended analysis timeframes. Acute stress responses may be analyzed in a 1-minute to 10-minute timeframe (e.g., 5-minutes) to and chronic mental health conditions may be analyzed in a 1-week to 4-week timeframe (e.g., 2-week).

The data modeling stage 304 implements a hierarchical approach to handle different temporal scales of chronic mental health assessment. The system 100 employs multiple machine learning models, each optimized for specific temporal resolutions, including short-term models focusing on acute stress and immediate emotional states, hourly models tracking pain, stress, exhaustion, and mood fluctuations, daily models monitoring sleep quality, gastrointestinal issues, and overall mood, and long-term models assessing chronic conditions aligned with clinical assessment timeframes. The model architectures include Random Forests for robust handling of heterogeneous feature sets, Gradient Boosting Machines optimized for temporal sequence prediction, Support Vector Machines particularly effective for stress state classification, and Multi-layer Perceptrons capable of capturing complex nonlinear relationships in physiological data.

The model tuning stage 305 implements a comprehensive optimization strategy using user-level cross-validation to maintain clinical validity and prevent data leakage. By way of example, data from approximately 85% of users are used for model development, with 5-fold cross-validation performed over users (i.e., training on ~80% of those users per fold and validating on ~20%). The remaining 15% of users are held out as an independent test set to evaluate generalizability. The feedback and optimization loops 306 enable continuous refinement of the entire pipeline, facilitating dynamic feature importance assessment, automated hyperparameter optimization, model performance monitoring, and adaptation to individual user characteristics. Performance metrics focus on both technical accuracy (e.g., using metrics such as normalized root mean squared error (NRMSE)) and clinical relevance (e.g., through correlation with validated clinical assessments). The system aggregates predictions over optimized time windows (as a non-limiting example, for a typical 10-20 minutes period) to align with the natural temporal scales of different mental health symptoms, ensuring robust processing of complex physiological data while maintaining clinical validity and enabling personalized mental health assessment across multiple temporal scales.

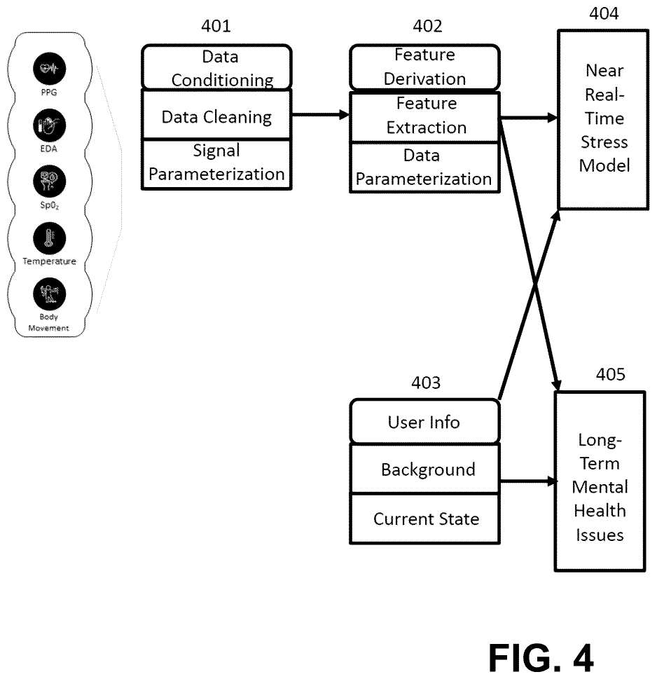

Referring to FIG. 4, the system 100 implements a dual-pathway architecture for simultaneously processing short-term and long-term mental health assessments through parallel modeling streams.

The architecture begins with a common data preprocessing foundation at the data conditioning, cleaning, and signal parameterization stage 401, which handles the complex multimodal biometric inputs from the wearable device. This stage implements sophisticated noise reduction algorithms specifically tailored for each biosignal type, including photoplethysmography (PPG), electrodermal activity (EDA), pulse oximeter, temperature, and body movement data. The cleaning processes employ adaptive filtering techniques that preserve physiologically relevant signal components while removing motion artifacts and environmental noise. For signals with different sampling frequencies, the system implements intelligent resampling strategies to ensure temporal alignment, with particular attention to maintaining the integrity of frequency-domain features critical for heart rate variability analysis.

The feature derivation, extraction, and data parameterization stage 402 processes the conditioned signals to extract clinically relevant indicators, as described herein.

The user information, background, and current state data input stage 403 incorporates contextual information critical for personalized mental health assessment. This includes demographic data, baseline physiological states, historical mental health records, and current self-reported conditions. The system 100 implements a dynamic knowledge representation that allows for continuous updates to user profiles while maintaining historical context. This stage also manages the administration and processing of self-report measures, including, but not limited to, the PHQ-9 for depression, GAD-7 for anxiety, PSS-10 for stress, BRS-6 for resilience, PCL-5 for PTSD, and SSS-8 for somatic symptoms.

The near real-time stress model 404 focuses on immediate stress detection and intervention. This model processes data in rolling windows of, e.g., 5-10 minutes, employing ensemble learning techniques that combine multiple classifier outputs for robust stress detection. The model architecture incorporates both gradient boosting machines for handling temporal sequences and support vector machines for acute stress classification. The real-time model maintains separate sub-models for different stress manifestations, including cognitive stress, emotional arousal, and physical tension, enabling targeted intervention recommendations.

The long-term mental health conditions model 405 implements a sophisticated hierarchical architecture for assessing chronic conditions across multiple timeframes. This model processes daily symptoms (sleep quality, gastrointestinal issues, mood), weekly patterns (somatic symptoms), and longer-term conditions (depression, anxiety, PTSD) using specific temporal aggregation strategies. This architecture employs deep learning components for capturing complex temporal dependencies, combined with traditional machine learning algorithms for interpretable feature importance analysis. The long-term model incorporates clinical validation metrics aligned with standard assessment periods (e.g., two weeks for depression and anxiety, four weeks for PTSD).

Both models implement continuous adaptation mechanisms through feedback loops that adjust model parameters based on user responses and outcome measurements. The system employs sophisticated cross-validation strategies, with 85% of data used for training (split 80-20 for training-validation) and 15% reserved for independent testing. The models are optimized using both technical metrics (NRMSE, ROC-AUC) and clinical correlation measures. Importantly, this architecture maintains separate pull-back models trained on reduced feature sets, ensuring system robustness when certain biosignals are unavailable. The entire dual-pathway architecture enables comprehensive mental health monitoring across multiple temporal scales while maintaining clinical validity and providing actionable insights for both short-term intervention and long-term mental health management.

It is understood that the above description is intended to be illustrative and not restrictive. The explanations and illustrations presented herein are intended to acquaint others skilled in the art with the invention, its principles, and its practical application.

Those skilled in the art may adapt and apply the invention in its numerous forms, as may be best suited to the requirements of a particular use. Many embodiments as well as many applications besides the examples provided herein will be apparent to those of skill in the art upon reading the above description.

Accordingly, the specific embodiments of the invention set forth herein are not intended as being exhaustive or limiting of the teachings. The scope of the invention should, therefore, be determined not with reference to the above description, but should instead be determined with reference to the appended claims, along with the full scope of equivalents to which such claims are entitled.

The omission in the following claims of any aspect of subject matter that is disclosed herein is not a disclaimer of such subject matter, nor should it be regarded that the inventors did not consider such subject matter to be part of the disclosed inventive subject matter.

The disclosures of all articles and references, including patent applications and publications, are incorporated by reference for all purposes.

Plural elements or steps can be provided by a single integrated element or step. Alternatively, a single element or step might be divided into separate plural elements or steps.

The disclosure of “a” or “one” to describe an element or step is not intended to foreclose additional elements or steps.

The use of “about” or “approximately” in connection with a range applies to both ends of the range. Thus, “about 20 to 30” is intended to cover “about 20 to about 30”, inclusive of at least the specified endpoints.

Unless otherwise stated, all ranges include both endpoints and all numbers between the endpoints in increments of one unit provided that there is a separation of at least 2 units between any lower endpoint and any higher endpoint. As an example, if it is stated that the amount of a component, a property, or a value of a variable such as, e.g., temperature, time, and the like is, e.g., from 1 to 90, from 20 to 80, or from 30 to 70, it is intended that intermediate range values such as, e.g., 15 to 85, 22 to 68, 43 to 51, 30 to 32, etc., are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest endpoint and the highest endpoint enumerated are to be considered to be expressly stated in this application in a similar manner.

The term “consisting essentially of” to describe a combination shall include the elements, components, or steps identified, and such other elements, components or steps that do not materially affect the basic and novel characteristics of the combination. The use of the terms “comprising” or “including” to describe combinations of elements, components, or steps herein also contemplates embodiments that consist essentially of the elements, components, or steps.

While the terms first, second, third, etc., may be used herein to describe various elements, these elements should not be limited by these terms. These terms may be used to distinguish one element from another element. Thus, a first element discussed herein could be termed a second element without departing from the teachings. Terms such as “first,” “second,” and other numerical terms when used herein do not imply a sequence or order unless clearly indicated by the context.

The flow charts described herein do not imply a fixed order to the steps, and embodiments of the present invention may be practiced in any order that is practicable, unless otherwise specified herein.

The terms “about,” “generally,” or “substantially” may account for a variation from the stated value of +/−10% or less, +/−5% or less, or even +/−1% or less. The terms “about,” “generally,” or “substantially” may account for a variation from the stated value of +/−0.01% or greater, +/−0.1% or greater, or even +/−0.5% or greater.

The method may comprise one or more of the steps recited herein. Some of the steps may be duplicated, removed or eliminated, rearranged relative to other steps, combined into one or more steps, separated into two or more steps, or a combination thereof.

### Appendix

The present teachings contemplate a system for multi-temporal mental health monitoring and intervention. The system may comprise a sensor network comprising a plurality of biometric sensors configured to collect physiological data including at least heart rate, electrodermal activity, respiratory rate, temperature, body movement, blood oxygen, and blood pressure measurements. The system may comprise a user device comprising a processor and a memory storing instructions that, when executed, process real-time physiological data from the sensor network. The system may comprise input/output devices coupled to the user device and configured to deliver assessment queries and receive user responses. The system may comprise a remote processing platform coupled to the user device and comprising processors and memory storing instructions that, when executed: may process the real-time physiological data through a multi-stage pipeline comprising data conditioning, feature extraction, and parallel model processing pathways; may execute a near real-time stress detection model for acute stress assessment; may execute a long-term mental health assessment model for chronic condition monitoring.

The data conditioning pathway may comprise: implementing piecewise linear, spline, and regression-based interpolation for missing values; performing adaptive filtering for noise reduction specific to each biosignal type; normalizing sampling frequencies across different physiological measurements; or any combination thereof.

The feature extraction may comprise computing: heart rate variability metrics including time-domain, frequency-domain, and nonlinear measures; electrodermal activity features including tonic and phasic components; respiratory sinus arrhythmia measurements using peak-valley methods; or any combination thereof.

The clinically validated assessment measures may comprise at least one of PHQ-9, GAD-7, PSS-10, BRS-6, PCL-5, and SSS-8 questionnaires.

The present teachings contemplate a computer program product for multi-temporal mental health monitoring, comprising: a non-transitory computer-readable medium storing instructions that, when executed by one or more processors, cause the one or more processors to: receive continuous streams of physiological data from a wearable sensor network; condition the physiological data by performing missing value imputation, outlier removal, and signal resampling; extract time-domain, frequency-domain, and nonlinear features from the conditioned physiological data; and/or process the extracted time-domain, frequency-domain, and nonlinear features through parallel modeling pathways comprising: a near real-time pathway implementing ensemble classifiers for acute stress detection, and a long-term pathway implementing hierarchical models for assessing chronic conditions; adapt model parameters based on user feedback and clinical validation metrics; generate interventional recommendations based on detected acute stress and chronic condition assessments; or any combination thereof.

The instructions may further cause the processors to: implement hierarchical model architectures comprising random forests, gradient boosting machines, support vector machines, neural nets, and multi-layer perceptrons; maintain separate sub-models for different mental health manifestations; aggregate predictions using ensemble learning techniques; or any combination thereof.

The adapting model parameters may comprise: performing cross-validation using training and validation data splits; optimizing model hyperparameters based on clinical correlation metrics; adjusting feature importance weights based on the user feedback; or any combination thereof.

The instructions may further cause the processors to: detect and classify different types of stress responses including cognitive, emotional, and physical manifestations; and/or generate targeted intervention strategies based on the classified stress type responses.

The present teachings contemplate a method for mental health monitoring and intervention implemented by at least one processor, comprising: receiving physiological measurements from a wearable sensor network including heart rate variability, electrodermal activity, respiratory, temperature, movement, blood oxygen, and blood pressure data; conditioning the received data through missing value interpolation, outlier removal, and frequency normalization; extracting features including statistical metrics, entropy measures, and nonlinear indices; processing the features through dual assessment pathways comprising executing real-time stress detection models, and executing long-term mental health assessment models; implementing model adaptation using feedback loops and clinical validation measures; maintaining pull-back model capabilities for scenarios with missing biosignals; generating personalized intervention recommendations based on detected mental health states; or any combination thereof.

The method may comprise calculating differential measurements for temperature and blood pressure; deriving activity state classifications from accelerometer data; computing moving averages for physiological signals using adaptive window sizes; or any combination thereof.

The generating personalized intervention recommendations may comprise: analyzing historical response patterns to different intervention types; considering current user state and environmental context; adapting recommendation timing based on detected stress levels; or any combination thereof.

The method may comprise: maintaining separate user profiles for different temporal assessment scales; updating baseline measurements based on longitudinal data analysis; adjusting model sensitivities based on individual response patterns; or any combination thereof.

### REFERENCE NUMERALS

100 system
101 sensor network
102 user device
103 remote processing platform
104 input/output (I/O) device
201 heart rate and heart rate variability (HRV) data
202 electrodermal activity data
203 respiratory data
204 temperature data
205 accelerometer data
206 pulse oximeter data
207 blood pressure data
301 data conditioning
302 data parameterization
303 feature extraction
304 data modeling
305 model tuning
306 feedback and optimization loops
401 data conditioning, cleaning, and signal parameterization stage
402 feature derivation, extraction, and data parameterization stage
403 user information, background, and current state data input stage
404 near real-time stress model
405 long-term mental health conditions model

1. A method for mental health monitoring and intervention comprising:
generating one or more biometric data sets from measuring and/or estimating one or more physiological markers via one or more biosensors;
extracting features from the one or more biometric data sets; and
executing parallel multi-temporal machine learning modeling using the extracted features as inputs, whereby the multi-temporal modelling includes:
a pipeline for one or more short-term models adapted for detecting acute stress responses; and a pipeline for one or more long-term models including:
hourly models adapted for detecting pain, stress, exhaustion, and mood fluctuations; daily models monitoring sleep quality, gastrointestinal issues, and overall mood; and models assessing chronic mental health conditions.

2. The method according to claim 1, wherein the one or more physiological markers include heart rate, heart rate variability, electrodermal activity, respiratory activity, body temperature, body movement, blood oxygen, blood pressure, or any combination thereof; and wherein each of the one or more biometric data sets corresponds with one of the one or more physiological markers.

3. The method according to claim 2, wherein the one or more physiological markers include at least heart rate variability.

4. The method according to claim 3, wherein the feature extraction for the heart rate includes statistical features including mean heart rate and standard deviation of heart rate.

5. The method according to claim 4, wherein the feature extraction for the heart rate variability includes statistical features, time-domain features, frequency-domain features, and nonlinear features; wherein the statistical features include mean heart rate variability and standard deviation of heart rate variability; wherein the time-domain features include standard deviation of NN intervals and root mean square of successive differences; wherein the frequency-domain features include normalized low-frequency, normalized high-frequency, and a ratio of normalized low-frequency to normalized high-frequency.

6. The method according to claim 4, wherein the nonlinear features are extracted by entropy analysis, nonlinear dynamic analysis, Poincaré plot analysis, second order data differential plotting, and chaotic modeling.

7. The method according to claim 2, wherein the feature extraction for the electrodermal activity accounts for both tonic and phasic components; and wherein the features include skin conductance level, skin conductance response, and a correlation coefficient between skin conductance level and time.

8. The method according to claim 2, wherein the features for the respiratory activity includes breath rate and respiratory sinus arrhythmia.

9. The method according to claim 2, wherein the features for the body temperature include mean temperature, standard deviation of temperature, and differential temperature over a time period.

10. The method according to claim 2, wherein the features for the blood pressure include a mean of blood pressure, a standard deviation of blood pressure, and differential blood pressure measured on a periodic basis.

11. The method according to claim 4, wherein the method further comprises, before the feature extraction, performing data parameterization of at least the heart rate variability including applying a moving average technique.

12. The method according to claim 1, wherein the long-term models include Random Forests, Gradient Boosting Machines, Support Vector Machines, and Multi-layer Perceptrons.

13. The method according to claim 1, wherein the short-term models include gradient boosting machines and support vector machines; and wherein the short-term models employ ensemble learning techniques.

14. The method according to claim 1, wherein the method further comprises administering one or more self-report measures.

15. The method according to claim 1, wherein the method further comprises, before the feature extraction, data conditioning of the one or more physiological markers; and wherein the data conditioning includes: implementing piecewise linear, spline, and regression-based interpolation for missing values; performing adaptive filtering for noise reduction specific to each type of the one or more physiological markers; and normalizing sampling frequencies accounting for different sampling frequencies of the one or more biosensors.

16. The method according to claim 1, wherein the method further comprises generating interventional recommendations based on the detected acute stress responses and/or the chronic mental health conditions.

17. A system for performing the method of claim 1, wherein the system comprises:
a sensor network comprising one or more biosensors;
a user device comprising one or more processors and one or more non-transitory memory storage mediums storing computer-readable instructions that, when executed, detect acute stress responses and/or detect chronic mental health conditions; and
a remote processing platform comprising one or more processors and one or more non-transitory memory storage mediums storing computer-readable instructions that, when executed, detect acute stress responses and/or detect chronic mental health conditions.

18. The system according to claim 17, wherein the one or more biosensors include at least a photoplethysmography sensor.

19. The system according to claim 17, wherein the one or more biosensors include one or more photoplethysmography (PPG) sensors, temperature sensors (e.g., thermocouples, resistance temperature detectors (e.g., negative temperature coefficient sensors), thermistors, or semiconductor-based integrated circuits), electrodermal activity (EDA) sensors, pulse oximeters, accelerometers, gyroscopes, or any combination thereof.

20. The system according to claim 17, wherein the sensor network is on-board a smart wearable device.
